(19) United States
(12) Patent Application Publication
(10) Pub. No.: US 2011/0268828 A1
(43) Pub. Date: Nov. 3, 2011

(54) CELL RECEPTOR BLOCKER FOR PREVENTING AND TREATING AVIAN INFLUENZA AND OTHER DISEASES CAUSED BY VIRUSES

(76) Inventor: Isidro Fernandez Siurob, Queretaro (MX)

(21) Appl. No.: 12/934,058

(22) PCT Filed: Mar. 30, 2009
(86) PCT No.: PCT/MX09/00027

§ 371 Date: Dec. 6, 2010

(30) Foreign Application Priority Data
Apr. 7, 2008 (MX) MX/A/2008/004777

(51) Int. Cl. A61K 35/76; A61K 35/74

(52) U.S. Cl. 424/780

(57) ABSTRACT

Use of previously inactivated viral particles of the avian influenza virus or of previously purified antigenic components thereof, alone or in combination with carriers of biological origin in a composition administered via the respiratory system in order to prevent and to treat avian influenza, and also the composition mentioned. The product inhibits the coupling of said pathogenic virus, occupying the cell receptors for viral haemagglutinins.

Avian influenza is an infectious disease caused by a type A orthomyxovirus, capable of infecting birds and mammals.

The enormous capacity for mutation and recombination of this virus, as well as the high pathogenicity that it can develop, represent a real threat to humanity.

Common flu, much less virulent, causes between 200,000 and 500,000 deaths per year, primarily among the elderly.

Avian influenza caused severe outbreaks in 2004 in South Korea, Japan, Thailand, Cambodia, Indonesia and Laos, causing the death or resulting in the sacrificing of approximately 120 million birds.

At present, it is possible to treat this disease with varying success by using various chemotherapies developed by the pharmaceutical industry. For its prevention, vaccines with relative efficacy against avian influenza have been developed.

### TECHNICAL FIELD OF THE INVENTION

Continual progress in immunology is producing more knowledge every day and in this field, I have developed a new product that is able to prevent and treat avian influenza and diminish the viral transmission by means of a different process.

### OBJECT OF THE INVENTION

The object of this invention is the prevention and treatment of diseases caused by viruses, such as avian influenza specifically, as well as other respiratory diseases whose route of access to the organism is the respiratory system, in a safe, rapid, widespread and effective way.

### PRIOR ART

At the date of filing of this application, it is not known that viral materials can be used to block cell receptors in order to prevent the infection of an organism by viruses or bacteria. Neither is it known that blocking receptors can be used for the treatment of viral or bacterial diseases.

It is likewise novel that this [solution] is proposed as a tool in the control of epizootics and epidemics in any species.

### TECHNICAL PROBLEMS TO BE SOLVED

At present, avian influenza is being prevented in birds by means of the application of inactivated or recombinant vaccines; unfortunately, the levels of immunoglobulins A (IgA) quickly decrease in the mucous membranes of the respiratory system, which allows for its becoming infected and the consequent viral replication and cell damage and death.

It is therefore necessary to quickly and appropriately prevent this from occurring.

It is for this reason that the technique of blocking receptors in this patent application was designed and developed.

### BRIEF DESCRIPTION OF THE INVENTION

The process consists in applying the product by aerial route in the form of an aerosol (spraying particles of 20 to 80 microns) in closed spaces when applied in widespread manner, that is, to groups of individuals, or by means of inhalers when applied in individual manner.

The product consists of previously inactivated virus particles or inactive parts of the virus itself or of its previously purified antigen components, alone or combined with vehicles of biological origin (recombinant) in an aqueous suspension that contains an adequate adjuvant to achieve contact of the product with respiratory epithelial cells, as well as with lymphoid cells associated with tissues of the respiratory system.

### DETAILED DESCRIPTION OF THE INVENTION

I describe the product as a RECEPTOR BLOCKER and its action consists in occupying, in the cells of the respiratory epithelium, the insertion sites of viral particles, preventing them from making contact, followed by penetration into the cell, thus eliminating cell damage and viral replication, diminishing the dissemination of infectious particles and the risk of new infections.

The product is used both in the prevention and the treatment of avian influenza, with the condition that treatment is applied upon the first symptoms of the disease.

The product is made up of previously inactivated viral particles or inactive parts of the virus itself or of its previously purified antigenic components, alone or combined with vehicles of biological origin (recombinant vehicles) in an aqueous suspension that contains an adequate adjuvant to achieve contact of the product with respiratory epithelial cells, as well as with the lymphoid cells associated with tissues of the respiratory system.

The coupling of the avian influenza virus to the host cells is followed by endocytosis and fusion of the virus with the endosomal membranes.

The product inhibits the coupling of the pathogenic virus by being presented before this coupling occurs and by occupying the cell receptors of viral hemoagglutinins that contain sialoligosaccharides on the cell surface.

The product is produced by replicating an avian influenza virus of low pathogenicity in embryonal chicken eggs. The virus is recovered from the amnioalantoid fluid and is inactive.

Components of Escherichia coli cell membranes are added to it and it is refrigerated until it is used.

The product does not cause secondary reactions of any kind, so that it is completely harmless.

The widespread form of application, given the characteristics of the product, makes possible its use for the containment of epizootics and epidemics, as well as the prevention and treatment of the same, since by making available and equipping adequate treatment areas, it is possible to treat hundreds or thousands of individuals at the same time, in only a few minutes.

### PRACTICAL EXAMPLES AND EVIDENCE

### Example A

The manner of executing its application is by immediate spraying when one suspects the appearance of an outbreak of avian influenza based on clinical symptoms. The person caring for the birds can apply the product to the birds suspected of having the illness at a rate of 25,000 birds an hour and administer the product four times in two days, once in the morning and once in the afternoon, which is the recommended administration for a better outcome.

### Example B

If a person in a population is diagnosed as being infected with an avian influenza pathogenic virus, the person can be isolated from the rest of the population and be administered the receptor “blocker”, keeping such persons together in a closed space where they will receive the blocker by inhaling an aerosol. This will prevent the infection and therefore the replication and dissemination of the disease, giving the public health authorities time to take additional steps for treatment and containment.

### ADVANTAGES AND DISADVANTAGES COMPARED TO THE PRIOR ART

1. There is no equal product in the world.
2. There is no similar product in the world.
3. It is the first product in the world that uses blocking of cell receptors to prevent and contain avian influenza and similar diseases.
4. It is not a chemotherapy or drug therapy.
5. It is harmless.
6. It is not a chemotherapy drug.
7. It can be applied in a widespread manner.
8. It can be applied in an individual manner.

Now that my invention has been described and its novelty pointed out, I claim as my exclusive property the content of the following clauses:

1. Blocking of cell receptors of the respiratory epithelium by the use and application of an aerosol, instillation, or any other medium, using materials derived from viruses or bacteria with the purpose of preventing, treating or reducing the dissemination of infectious diseases of animals and man, especially avian influenza.

2. The product described and its use for blocking of receptors.
